(19) United States
(12) Patent Application Publication
(10) Pub. No.: US 2010/0192408 A1
(43) Pub. Date: Aug. 5, 2010

(54) Footwear sole with anti-stress and slight massaging effect

(75) Inventor: Lucio Righetto, Camponogara (VE) (IT)

Correspondence Address: Themis Law, 7660 Fay Ave Ste H-535, La Jolla, CA, 92037, US

(73) Assignee: BALLY SHOE FACTORIES, Caslano (CH)

(21) Appl. No.: 12/064,340

(22) PCT Filed: Jul. 3, 2006
(86) PCT No.: PCT/EP2006/063820

§ 371 Date: Jun. 3, 2008

(30) Foreign Application Priority Data
Jul. 4, 2005 (IT) SV2005A000024

(51) Int. Cl. A43B 13/20; A43B 13/12; A43B 13/38; A43B 13/18; A61F 5/14; A43D 8/00

(52) U.S. Cl. 36/29; 36/30 R; 36/44; 36/28; 36/141; 12/146 B

(57) ABSTRACT

Footwear sole with anti-stress and slight massaging effect comprising an external sole (1) resting on the ground and at least an inner sole, interposed between said external sole and the user foot, which inner sole is composed of at least two layers a first layer directly contacting the foot, so called cleaning insole (4) and a second layer placed between said cleaning insole and said external sole (1) called intermediate layer (2), and in which said intermediate layer (2) is at least partially composed of at least a soft layer (3) made of soft material, said cleaning insole (4) having at least partially slots (104) through which said soft layer (3) penetrates and contacts the foot. The present invention relates also to a footwear footbed with anti-stress effect comprising a first lower layer (42) and a second upper layer (41) and wherein said first (42) and said second (41) layer are at least partially overlapped to form said footbed (40), said first (42) and said second (41) layer being made of soft materials.

The present invention relates to a footwear sole with anti-stress and slight massaging effect comprising an external sole for resting on the ground and at least an inner sole interposed between said external sole and the user foot, which inner sole is composed of at least two layers a first layer directly contacting the foot, so called cleaning insole, and a second layer placed between said cleaning insole and said external sole called intermediate layer.

These type of soles are known and widely used. While such soles satisfactorily serve their function, they still suffer from certain drawbacks.

At present soles according to prior art have an arrangement of separated anti-stress members providing the presence of some anti-stress members in the footwear area corresponding to the user foot. The presence of these individual anti-stress inserts leads to certain drawbacks: when the footwear is used for a long time, or it is subjected to a high stress, such as for example footing footwears or it is subjected to a high weight of the user, or when the user walking is not anthropometrically correct, individual inserts tend to be oriented in different ways one with respect to the other, sometimes opening like a fan or tilting, reducing the anti-stress effect and absorption of micro-shocks suffered from the foot when walking. The user weight is not uniform on all the foot sole, and consequently the footwear, but it has areas where the weight is greater and areas where the force is less concentrated. Moreover the walking process has a vectorial distribution of forces that is very complex and variable in each moment of walking, with a force distribution on the foot sole following cylic type processes, repeating at each step of the user.

The sequence of micro-shocks suffered from the foot each time it contacts the ground, that is substantially at each step, is the main cause of foot stress and user tiredness.

In addition plantar reflexology principles identify different foot areas, that if properly stimulated cause tiredness or relaxation for the user. Common footwear soles do not have separated and distinct slight massaging areas based on plantar reflexology principles, but substantially just provide separated anti-stress members positioned in areas selected according to their location on the sole based on the concept of limiting above micro-shocks.

It is therefore important to have a footwear sole that besides the fact of having generically positioned anti-stress members, it provides the position of these anti-stress members in specific and definite areas of the foot sole, based on plantar reflexology principles.

In addition still according to prior art soles said separated anti-stress members are “embedded” in a stiff sub-layer, usually called mounting insole, that therefore provides a considerable stiffness to the footwear, that does not have an optimal bending during walking, and so partially it makes fruitless the result that can be obtained by these individual anti-stress members embedded in a stiff sub-layer, with which the user foot comes in contact, this is due also to the different orientation that individual anti-stress members tend to take during use, whether for a usual use or related to particular stresses.

A further problem in prior art soles is related to the fact of manufacturing the footwear having individual anti-stress members, and it is due to the fact that they have to be made separately along the plan dimensions of the sole, causing an increase in cost for manufacturing the footwear having said members.

Moreover prior art soles tend to move away from the user foot at least partially when walking and especially in the plantar arch area, which tipically is not completely in resting position, causing a weight distribution that is concentrated in the heel area and in the tip area of the foot, particularly in the front area and surrounding and/or corresponding to the bending line, that is the metatarsus area, where the foot naturally bends when walking.

Another drawback related to prior art footwears is due to the fact that materials used for the inner sole of the footwear often do not have a suitable comfort for the user, mainly due to material density, that is not optimal for comfort, since flexibility, and density of the material are often adjusted to offer a considerable strength of the sole, and so they are too stiff for guaranteeing a high comfort.

Aim of the present invention is to provide a footwear sole with anti-stress and slight massaging effect overcoming drawbacks of known footwear soles in a simple and inexpensive way.

The invention achieves above aims by providing a footwear sole with anti-stress and slight massaging effect comprising an external sole for resting on the ground and at least an inner sole interposed between said external sole and the user foot, which inner sole is composed of at least two layers a first layer directly contacting the foot, so called cleaning insole, and a second layer placed between said cleaning insole and said external sole called intermediate layer, and wherein said intermediate layer is at least partially composed of at least a soft layer made of soft material, said cleaning insole at least partially having slots through which said soft layer penetrates and contacts the foot.

According to the present invention considerable advantages are obtained overcoming prior art drawbacks, the soft layer made of soft material contacts the foot through said slots producing a slight massage and anti-stress and micro-shock absorbing effect causing a beneficial result on the user foot and on the user, reducing felt tiredness and stress.

Advantageously the footwear sole according to the present invention is provided to have different embodiments.

According to a first preferred embodiment the soft layer extends only for a part of the plan dimensions of the inner sole, the remaining, complementary part of said inner sole being made of a stiffer material layer, and it makes a mounting insole. In this case drawbacks related to prior art soles due to the fact of having individual anti-stress members made in a stiff sub-layer can be overcome. According to the above embodiment the soft area, accomplishing the anti-stress effect is as an alternative to or complementary to the stiff area, that is the mounting insole, actually making a sole having an alternation of stiff areas and soft areas, carrying on a beneficial slight massaging and anti-stress effect on the user foot, absorbing micro-shocks related to walking only where it is necessary. Particularly it is possible to provide said slots on said cleaning insole to be made in the area of said cleaning insole substantially coinciding with said soft layer, and said slots to be arranged according to any predetermined pattern in the area of said cleaning insole substantially coinciding with said soft layer. According to a preferred and advantageous variant embodiment the soft layer extends substantially in the area of the foot tip from the bending point of the foot to substantially the end of said inner sole corresponding to the foot tip, that is in the substantially front area coinciding with the foot metatarsus. This area is the most stressed one when walking since it is the area where the user applies the greatest stress when making the step, and therefore it is the one where not only the stress directly related to supporting the body weight is applied but also the stress related to pushing forwards the user body. By making a footwear sole according to the present embodiment the force necessary to push forwards the body is therefore unloaded by the user on a soft resting surface, accomplishing at the same time the massaging action of the user foot by means soft material coming out from said slots due to compression and squashing of the area.

According to a further variant embodiment it is possible to provide two, three or more soft layers, particularly two soft layers, a first soft layer from the bending area of the foot to substantially the end of said inner sole corresponding to the foot tip, and a second soft layer at the area of the plantar arch. Thus advantageously even the slight massage of the plantar arch area is obtained with a soft area, accomplishing a further and advantageous slight massaging effect, absorbing at the same time also micro-shocks when the foot rests during walking.

Advantageously slots are made on the cleaning insole from the bending area of the foot to substantially the end of said inner sole corresponding to the foot tip, and/or at the plantar arch area, according to any pattern, thus the soft material, coming out from said slots and contacting the foot helps in increasing comfort for the user both during the step when the foot moves away from the ground, and during the step when the foot rests on the ground.

According to a further preferred embodiment the intermediate layer is composed of at least a single layer of soft material, so called soft layer, extending substantially without interruption along the plan dimensions of said inner sole. Thus an advantageous and efficient absorbing action of micro-shocks on all the foot sole is obtained. The intermediate layer has at least partially along all the plan dimensions of said inner sole a further layer that is stiffer with respect to the soft layer placed under said soft layer, so called lower mounting insole. Thus the sole according to the present invention provides to absorb micro-shocks all along the foot sole and at the same time it has continuous and discontinuous areas wherein there is a stiff insole helping in increasing strength of the footwear, without affecting comfort.

It is also possible to provide the lower mounting insole to extend all along the plan dimensions of said inner sole, advantageously carrying on the function of increasing the footwear strength, or it is possible to provide the lower mounting insole extending along part of the plan dimensions of said inner sole, and advantageously extending along the part of the plan dimensions of said inner sole substantially coinciding with the area of the inner sole coming from the bending area of the foot to the foot heel. In this area it is preferable for the strength of the footwear to be greater, since this area is the first area contacting the ground when walking. Advantageously slots are made on the cleaning insole from the bending area of the foot to substantially the end of said inner sole corresponding to the foot tip, and/or at the area of the plantar arch, according to any pattern. Thus a greater comfort of the foot area moving away as the last one from the ground when walking and upon which the stress pushing forwards the body is generated.

Advantageously it is possible to provide slots to be made on all said cleaning insole and/or slots to have a different arrangement with areas with a greater density of slots and areas with a lower density of slots, such to distribute the slight massage in areas of greater interest according to plantar reflexology principles.

According to a further advantageous characteristic of the present invention it is possible to provide one or more padding or cushioning areas as an alternative or in combination with the soft layer.

According to an advantageous embodiment at least one of said cushioning area is made at the bending area of the foot, that due to anthropometric reasons hardly supports the walking stress. By the above embodiment the presence of a cushioning area helps in increasing user comfort. Particularly said cushioning area made at the bending area of the foot is provided to be in direct contact with the foot, such to make at best its absorbing shock functions and comfort functions, at the same time enlarging the resting area of the foot during bending when walking, and making a resting area that is not only larger but also softer than prior art footwears.

Moreover it is advantageously possible to provide at least one of said cushioning area to be made at the foot heel, and particularly to be placed under said inner sole, at the part opposite to the inner sole one contacting the foot. Thus the micro-shock when the foot rests is almost totally absorbed by the cushioning area and only subsequently it is transmitted to the remaining part of the sole.

According to advantageous embodiment solutions it is possible to provide said slots to be different one with respect to the other with regard to shape and size depending on the fact of corresponding with different areas of the foot sole, in order to apply plantar reflexology principles to the sole according to the present invention. Particularly said slots are provided to be identical one with respect to the other as regards shape and size, with a circular plan shape.

The soft layer is preferably made of gel, polyurethanes, rubbers or like soft materials, coming out from slots when the foot squeezes the corresponding footwear or sole area.

As an alternative it is possible to provide said soft layer to project by a predetermined amount from said slots coming into contact with the foot, even without a further stress of the user weight.

Particularly and advantageously the soft layer is made of materials injectable according to conventional injection methods.

Padding or cushioning areas are advantageously preferably made of anti-shock material like Poron®(licensed trademark) and/or of gel, polyurethanes, rubbers or like soft materials, such to optimally absorb micro-shocks when walking.

According to a further preferred variant embodiment, in order to advantageously make the footwear more soft and comfortable the sole is provided to have a seam between said inner sole and the footwear lining, preferably in the area corresponding to the area from the bending area of the foot substantially to the end of said inner sole corresponding to the foot tip.

A further object of the present invention is a footwear sole with anti-stress and slight massaging effect comprising an external sole resting on the ground and at least an inner sole, interposed between said external sole and the user foot wherein said inner sole is completely in contact with all the foot sole when walking. By the soft layer and/or the cushioning areas the sole according to the present invention is always in contact with the foot sole of the user, during all walking steps, for example avoiding the plantar arch to be moved away from the sole, and thus avoiding the subsequent micro-shock corresponding to the contact of the plantar arch with the sole that occurs when walking. If the plantar arch moves away from the sole when the foot raises, when the foot is again in contact with the ground the plantar arch again contacts the sole, actually causing a micro-shock affecting the comfort felt by the user.

According to a further advantageous characteristic of the present invention it is possible to provide said inner sole to be movable from said external sole and to be used individually for adding an anti-stress and/or slight massaging inner sole in already manufactured footwear, in order to increase comfort, or said inner sole to be made together with the footwear in order to produce a footwear with anti-stress and/or slight massaging effect.

A further object of the present invention is that of providing a method for manufacturing a sole according to the present invention overcoming known drawbacks of prior art processes.

A further object of the present invention is a footwear footbed with anti-stress effect comprising a first lower layer and a second upper layer and wherein said first and second layer are overlapped to form said footbed, said first and said second layer being made with soft materials.

Said second upper layer is intended to be arranged inside the footwear faced towards the user foot, while said first lower layer is intended to be faced towards the ground. Therefore terms upper and lower are to be considered referring to the correct mounting position of the footbed inside the footwear.

Advantageously the ratio between density of the first lower layer and said second upper layer is

(lower layer density/upper layer density)=0.8-0.9

particularly it is preferable that

(lower layer density/upper layer density)=0.85(+/−0.01)

Such density ratio produces a considerable comfort to the user foot, the combination of these two layers having such density ratio cause an optimal absorption of shocks due to walking action and at the same time it produces also a pleasant massage to the foot sole of the user.

Particularly the upper layer has a density greater than the lower layer and a sort of shape memory, and that allows the footwear to adapt to the shape of the user foot and also to adapt to particular stresses exerted by the user foot on the footbed when walking. It is evident that each person, different as regards physical conformation and way of walking, weight and dimensions, exerts on the footwear a different distribution of forces when walking.

The presence of an footbed according to the present invention allows the footwear to adapt to each different distribution of weights and pressures due to different users.

Particularly this advantageous characteristic is obtained by using two different materials for said first and said second layer, that is the layer intended to be faced towards the footwear sole and/or towards the ground when walking, i.e. the lower layer is preferably made of synthetic material, particularly synthetic rubber, so called TR rubber adding pharmaceutical oils.

TR rubber is known to person skilled in the art and substantially it is a thermoplastic, synthetic rubber with a lower cost and good mechanical characteristics, in this present invention it is preferred to use TR rubber with a SHORE hardness from 2 to 9 degrees, particularly from 3 to 8 degrees, preferably 5+/−2 degrees.

Particularly the lower layer advantageously has the following characteristics:

| Technical | | Measurement | Average |
|---|---|---|---|
| characteristics | Method | unit | value |
| Density | DIN 53479 | gr/cm³ | 0.86 +/− 0.01 |
| | Method A | | |
| Tensile | DIN 53504 | Kg/cm² | 14 +/− 2 |
| Strenght (at | Test samp. S1 3 | | |
| break) | | | |
| Elongation (at | Din 53504 | % | 1050 +/− 50 |
| break) | test samp S1 3 | | |
| | Mm thickness | | |
| | trac sp. 500 | | |
| Melt Flow Index | DIN 53735 | Gr/10 min. | 200 value |
| | | | approx |
| Rebound | DIN 53512 | % | 15 +/− 4 |
| resilience | | | |
| Hardness | Din 53505 | Shore A | 5 +/− 2 |
| Compression set | ISO 815:1991 | 70° C. 24 h % | 40 +/− 5 |
| | (E) | | |
| | | 55° C. 24 h % | 5 +/− 2 |
| | | 55° C. 48 h % | 5 +/− 2 |
| | | 55° C. 72 h % | 5 +/− 2 |

The upper layer, that is the layer of the footbed faced towards the user foot and at least partially overlapped to the above lower layer, is advantageously made all or partially by a synthetic material, and preferably by a synthetic material based on polyurethane, characterized in that it has specific chemical-physical characteristics making it particularly comfortable and strong.

Particularly the synthetic material based on polyurethane is characterized in that it has following characteristics: dimensionally stable, good absorption of shear forces, good buffering capacity, good pressure distribution, non-irritating to the skin, high elasticity, high shock absorbing power, it adheres well with glue, unplasticised and light.

In order to make clear to person skilled in the art preferred technical characteristics of the material used for the upper layer are the following:

| Test | | TG | | | |
|---|---|---|---|---|---|
| properties | Unity | method | Soft | Medium | Hard |
| Tensile | kPa | DIN | 140-200 | 200-300 | 300-400 |
| strength | | 53571 | | | |
| Elongation | % | DIN | 900-700 | 700-400 | 400-250 |
| at break | | 53571 | | | |
| Shore A | / | DIN | 0-5 | 5-10 | 10-20 |
| | | 53505 | | | |
| Shore L | / | DIN | 5-14 | 14-25 | 25-40 |
| | | 53519 | | | |

| | | | |
|---|---|---|---|
| Density | Kg/m³ | DIN | 1006 |
| | | 53479 | |

Moreover preferably the lower layer is discontinuous, and it is at the foot sole area and heel area, and it has one or more slots, while the upper layer is continuous or at least partially continuous in areas corresponding to said lower layer.

Particularly slots in the lower layer are advantageous for comfort, since they produce a kind of escape for the upper layer. The upper layer and the lower layer of the footbed are pressed together during the walking action or movement by the user, and squashed together. Slots in the lower layer allow the upper layer to project through said slots towards the part opposite to the user foot, increasing pliability of footbed areas and so of the footwear wherein said footbed is mounted, wherein pressure and forces due to walking action are exerted. In practice when the user rests the weight in the footbed, the upper layer gives way and it compresses itself downwardly, meeting the lower layer, which lower layer in turn is elastically compressed downwardly, and it produces an escape, at least partially, by slots for elastic deformation of the upper layer. Thus the user feels a greater comfort and softness.

Moreover it is possible to provide a gap between said upper layer and said lower layer, that is an air cushioning area, helping in increasing comfort.

Buffering capacity of this particular type of upper layer described above is such that the upper layer adapts itself to the shape of the user foot, shaping itself about the foot as it were, but it has such a density to allow a considerable comfort of the user foot, while maintaining elasticity suitable to comfort. So, at each step, or even only due to weight rests of the footbed, the footbed shapes itself around the foot and it allows the footbed to adhere to all the foot sole of the user. The buffering capacity of the upper layer is realized in an elastic recovery of the upper layer, that is not immediate as the elastic recovery of the lower layer, but is has a dormant time allowing to feel a considerable comfort above all in more stressed areas.

It is also possible to provide the application of a further cushion for absorbing shocks in the area substantially corresponding to the bending area of the foot, in order to optimize user comfort.

Said cushion for absorbing shocks may be made of one of the two material described above or of conventional materials and it may be made integral with said first layer or said second layer or it may be applied subsequently.

Preferably said cushion for absorbing shocks is made of the same material as the upper layer and is applied upon said upper layer, in the area substantially coinciding with the bending area of the foot immediately before the plantar arch area.

The footbed according to the present invention is preferably intended to be mounted in a footwear and to be overlapped to a mounting insole, tipically made of texon and/or fibres, which mounting insole is overlapped to the footwear sole, and it can be covered by a conventional cleaning insole, which cleaning insole directly contacts the user foot.

As an alternative said insole according to the present invention can be mounted also on already manufactured footwears, or as an alternative to or in combination with the mounting insole and/or cleaning insole if they are not present.

The process according to the present invention comprises following steps:

A—providing a footwear shell made of at least an external vamp and an inner lining

B—providing an external sole

C—providing an inner sole, said inner sole being made of at least a first layer directly in contact with the foot, so called cleaning insole,

D—providing a second layer of the inner sole, so called intermediate layer at least partially composed of a layer of soft material, called soft layer,

E—providing slots on said cleaning insole at least partially along the plan dimensions of the insole corresponding to the soft layer through which said soft layer comes in contact with the foot,

F—assembling said first and said second layer to form said inner sole by sewing and/or gluing and/or joining and/or chemical physical bonding.

G—assembling said inner sole with said external sole and with said footwear shell by sewing and/or gluing and/or joining and/or chemical physical bonding.

According to this process the footwear is assembled starting from a sole having anti-stress and slight massaging effect.

According to a preferred variant embodiment of the process, it comprises following steps:

A—providing a footwear shell composed at least of an external vamp (98) and a inner lining

B—providing an external sole

C—providing an inner sole, said inner sole being made of at least a first layer directly contacting the foot, so called cleaning insole,

D—providing slots on said cleaning insole at least partially along the plan area of the insole corresponding to the soft layer

E—assembling said inner sole with said external sole at peripheral edges by sewing and/or gluing and/or joining and/or chemical physical bonding.

F—between said inner sole and said external sole, in areas of the inner sole wherein said slots are provided injecting a soft material, such as gel, polyurthanes, rubbers or like soft materials, making a second layer of the inner sole

G—assembling said inner sole and/or said external sole and/or said footwear shell by sewing and/or gluing and/or joining and/or chemical physical bonding.

Advantageously according to this process the soft or tender material is injected by means of known injection processes inside the partially manufactured sole, generating a considerable saving in costs and manufacturing time.

Moreover it is possible to provide the step of providing a footwear shell made of at least an external vamp and an inner lining and a reinforcement section interposed between said external vamp and said inner lining; thus the footwear is advantageously more strong and does not have heavy manufacturing costs.

According to an advantageous variant of the process for manufacturing a footwear according to the present invention there is provided a sewing and/or gluing and/or joining and/or chemical physical bonding for said lining with said soft layer at the peripheral area of the inner sole from the bending area of the foot to substantially the end of said inner sole corresponding to the foot tip. Thus the footwear obtained in this way has a still greater flexibility, since various parts of the footwear are free to slide if they are not engaged one with the other, leading to a footwear having the greatest comfort and flexibility. Therefore advantageously it is possible to provide to leave the external vamp and the inner vamp mechanically disengaged for at least a part or for all remaining parts of their surface dimensions.

In combination with above processes it is possible to provide further steps related to the fact of providing variant embodiments of the present invention.

Moreover since it is provided to make also a sole that can be inserted in an already manufactured footwear, also a method and a process comprising following steps are object of the present invention:

a—providing an external sole

b—providing an inner sole, said inner sole being made of at least a first layer directly contacting the foot, so called cleaning insole,

c—providing a second layer of the inner sole, so called intermediate layer at least partially composed of at least a layer of soft material, called soft layer,

d—providing slots on said cleaning insole at least partially along the plan dimensions of the insole corresponding to the soft layer through which said soft layer comes in contact with the foot,

e—assembling said first and said second layer to form said inner sole by sewing and/or gluing and/or joining and/or chemical physical bonding.

f—assembling said inner sole with said external sole by sewing and/or gluing and/or joining and/or chemical physical bonding.

Particularly according to a preferred variant embodiment there is provided a process for manufacturing a sole characterized in that it comprises following steps:

a—providing an external sole

b—providing an inner sole, said inner sole being made of at least a first layer directly contacting the foot, so called cleaning insole,

c—providing slots on said cleaning insole at least partially along the plan dimensions of the insole corresponding to the soft layer,

f—assembling said first and said second layer at peripheral edges by sewing and/or gluing and/or joining and/or chemical physical bonding.

g—between said inner sole and said external sole, in areas of the inner sole wherein said slots are provided injecting a soft material, such as gel, polyurthanes, rubbers or like soft materials, making a second layer of the inner sole

h—assembling said inner sole and/or said external sole by sewing and/or gluing and/or joining and/or chemical physical bonding.

Even in this case, like the process for manufacturing the footwear, the step injecting the soft or tender material allows a considerable saving as regards manufacturing time and costs.

Further characteristics and improvements are object of claims.

Characteristics of the invention and advantages derived therefrom will be more clear from the following detailed description of annexed figures, wherein:

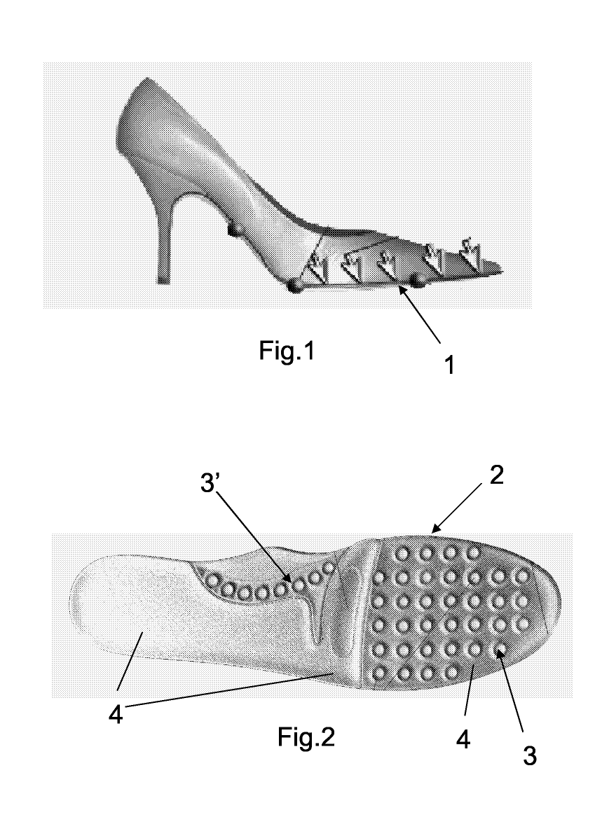

FIG. 1 is a side view of a footwear according to the present invention;

FIG. 2 is a plan view of an embodiment of the sole according to the present invention;

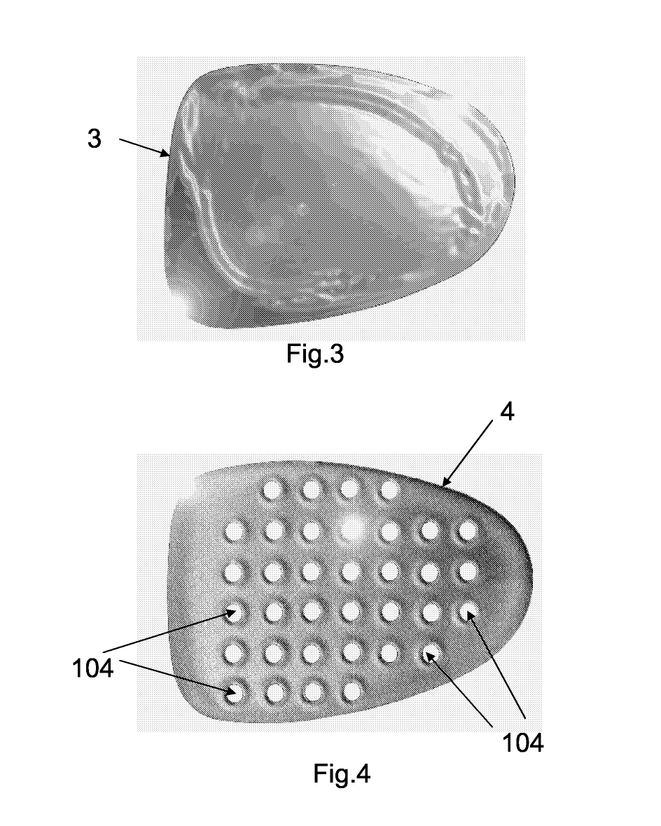

FIG. 3 is a plan view of a soft layer of the sole according to the present invention;

FIG. 4 is a plan view of a cleaning insole of the sole according to the present invention;

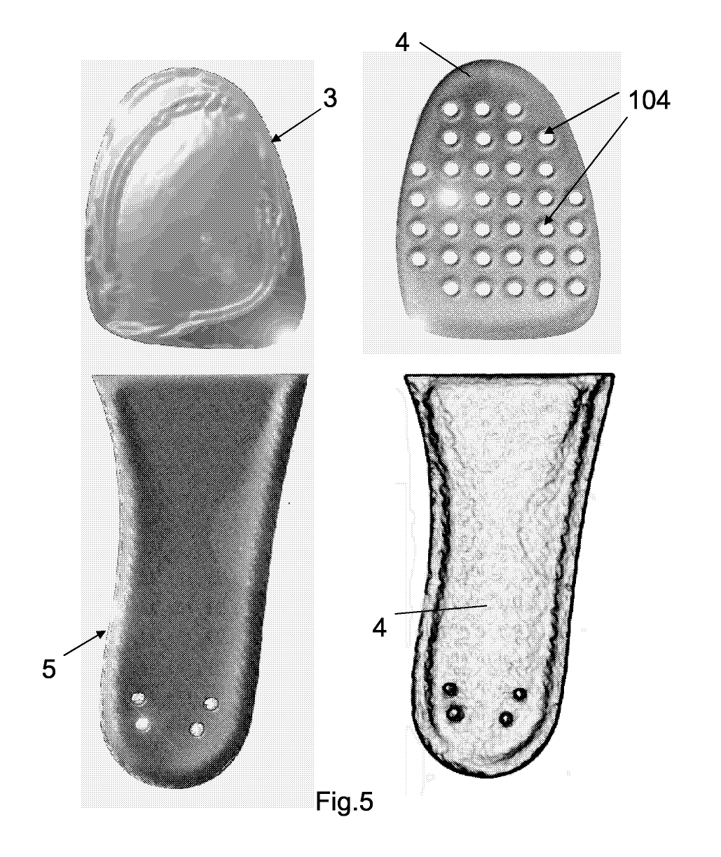

FIG. 5 is a plan view of various parts constituting a preferred embodiment of the sole according to the present invention;

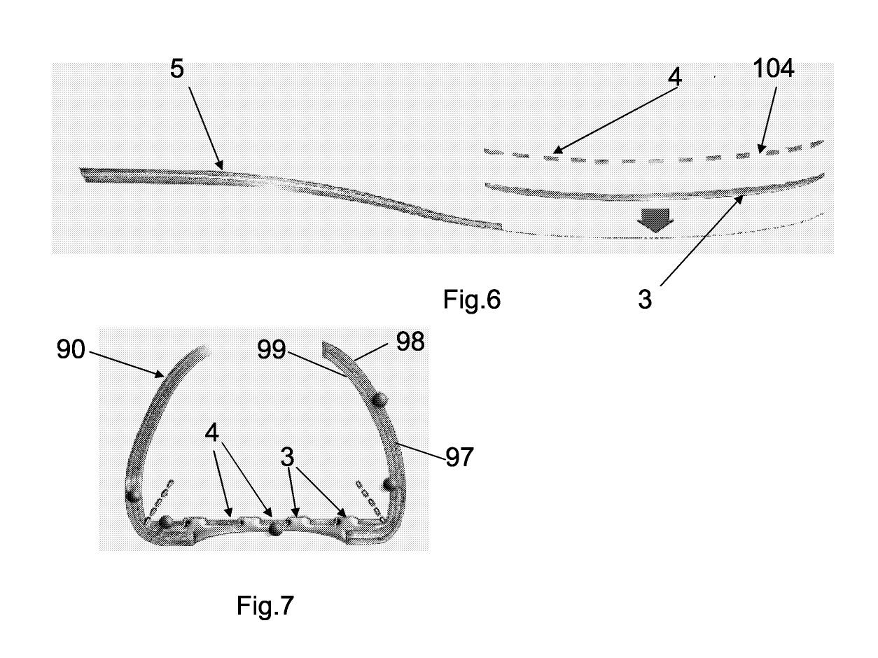

FIG. 6 is a side view of various parts constituting a preferred embodiment of the sole according to the present invention;

FIG. 7 is a section view of various parts constituting a preferred embodiment of the footwear and of the sole according to the present invention;

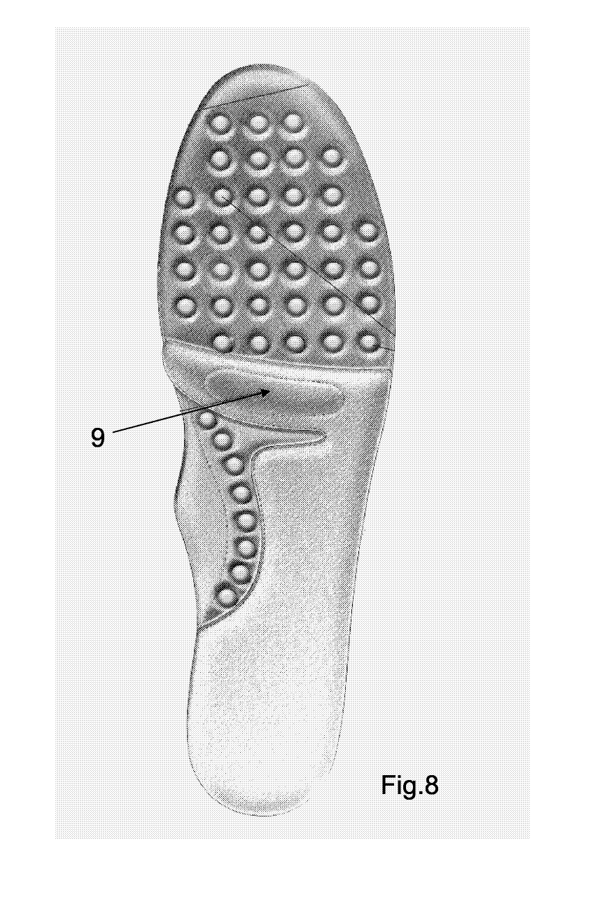

FIG. 8 is a plan view of a preferred embodiment of the sole according to the present invention;

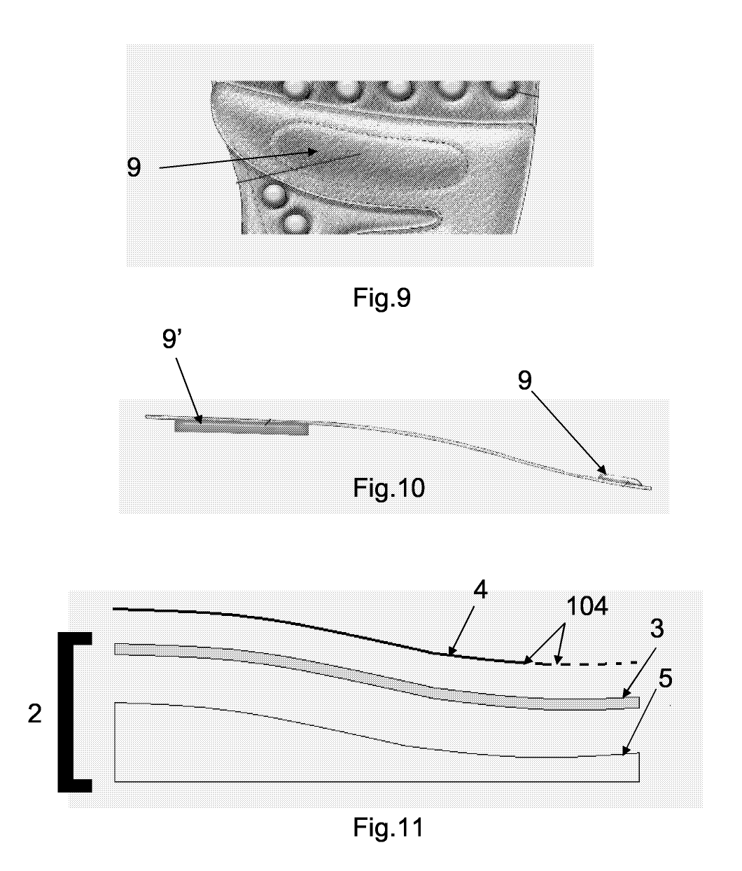

FIG. 9 is a plan view of an embodiment detail of the sole according to the present invention;

FIG. 10 is a section view of an embodiment detail of the sole according to the present invention;

FIG. 11 is a side view of various parts constituting a further preferred embodiment of the sole according to the present invention.

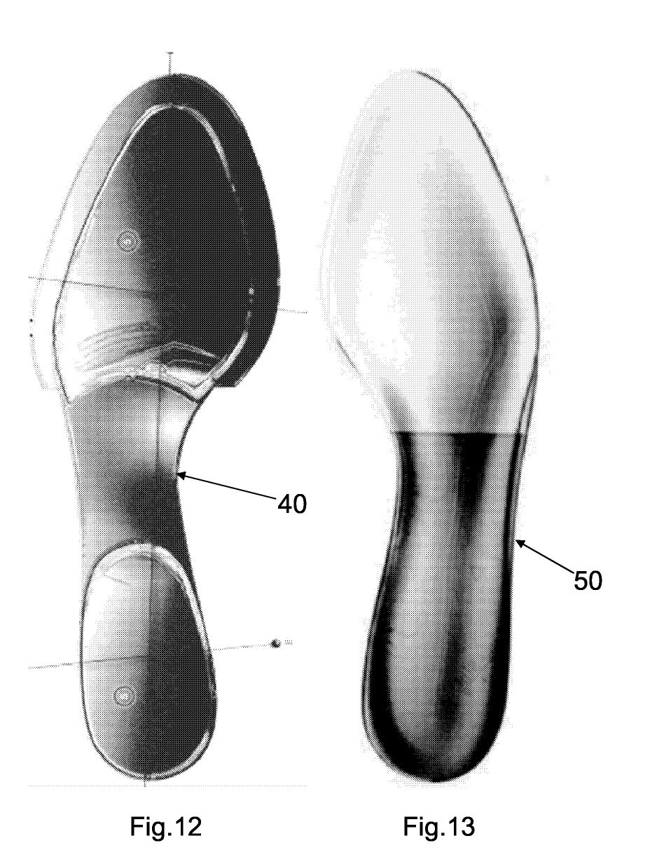

FIG. 12 is a top plan view of a footbed according to the present invention;

FIG. 13 is a top plan view of two insoles overlapping each other by way of an example;

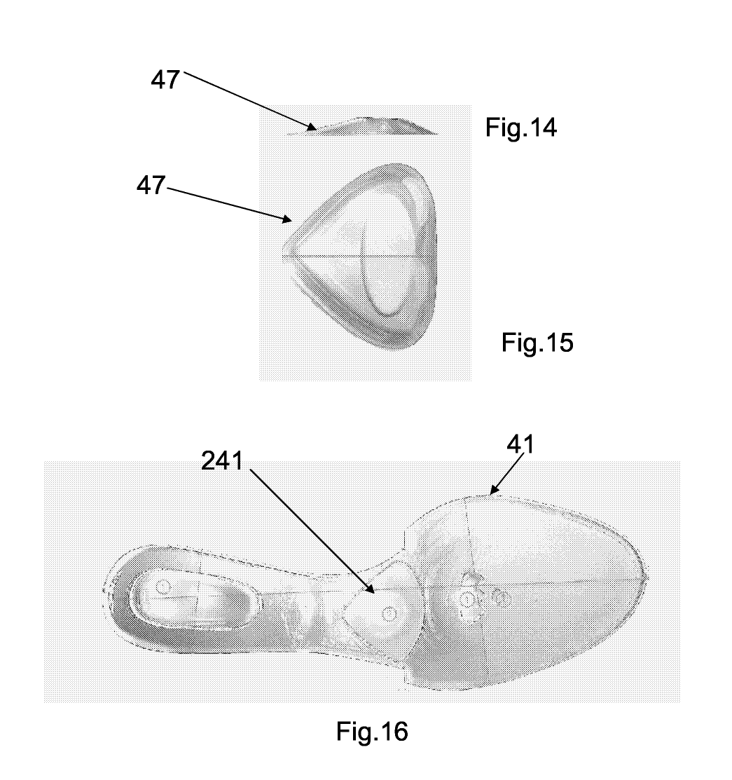

FIG. 14 is a section view of a cushion for absorbing shocks according to the present invention;

FIG. 15 is a plan view of a cushion for absorbing shocks according to the present invention;

FIG. 16 is a plan view of the upper layer of said footbed according to the present invention with the preferred mounting position of the cushion for absorbing shocks;

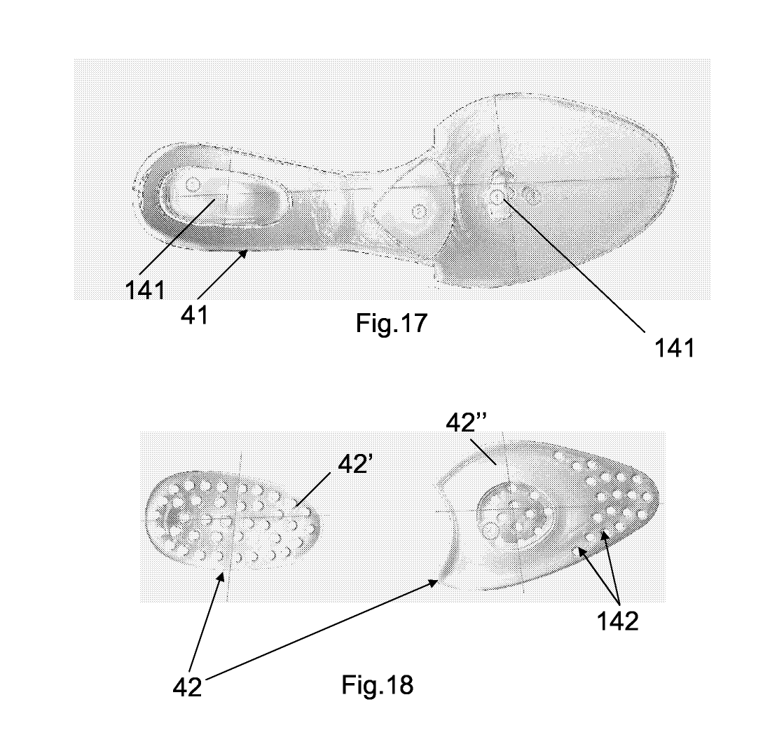

FIG. 17 is a plan view of the upper layer of the footbed according to the present invention;

FIG. 18 is a plan view of the lower layer of the footbed according to the present invention;

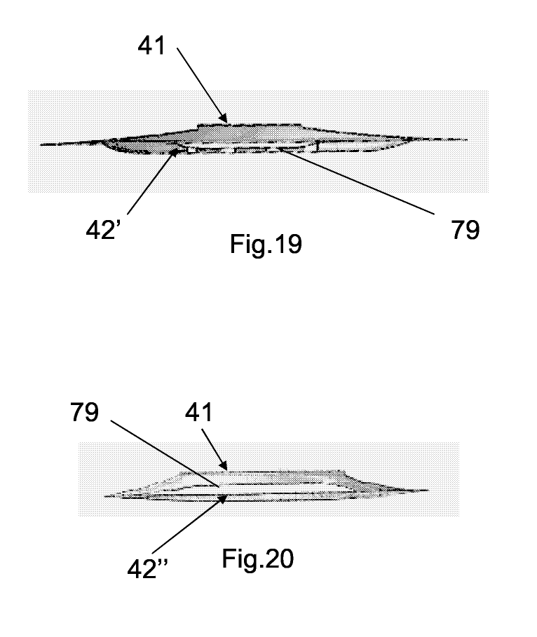

FIG. 19 is a cross-section view of the lower layer and of the upper layer of the footbed according to the present invention in the area of the footbed foot sole;

FIG. 20 is a cross-section view of the lower layer and of the upper layer of the footbed according to the present invention in the area of the footbed heel;

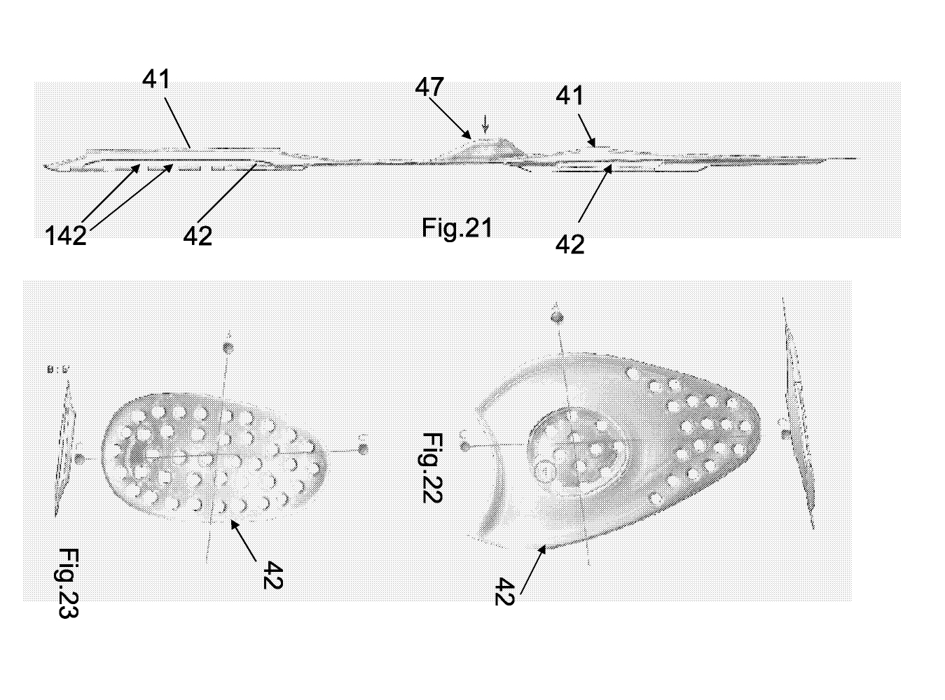

FIG. 21 is a longitudinal section view of the lower layer and of the upper layer of the footbed according to the present invention;

FIG. 22 is a comparison of the plan view of the lower layer and of the section of the lower layer at the front area or insole sole according to the present invention;

FIG. 23 is a comparison of the plan view of the lower layer and of the section of the lower layer at the rear area or insole heel area according to the present invention;

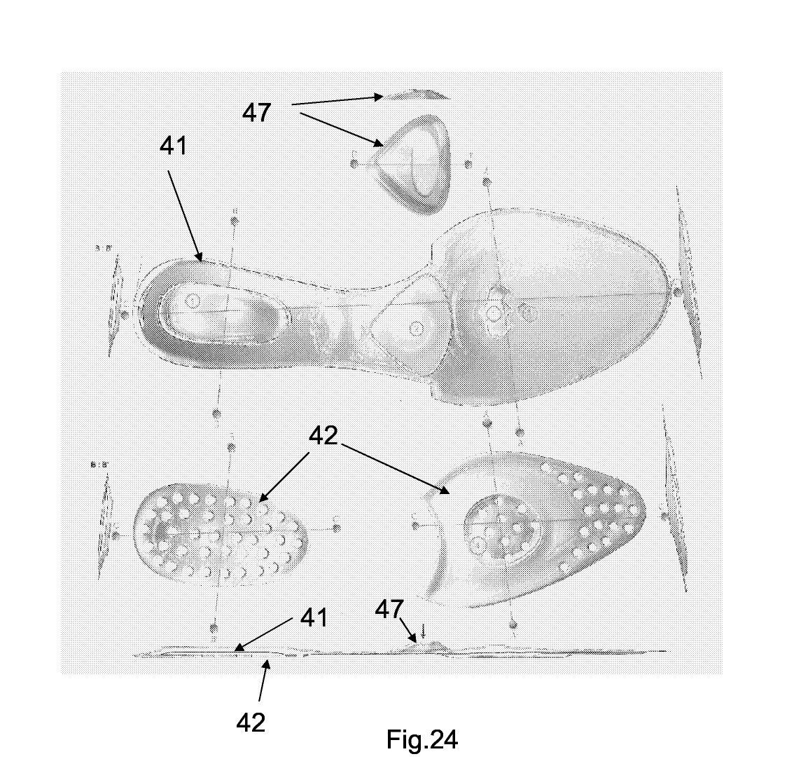

FIG. 24 is an exploded view and sections of the footbed according to the present invention.

The sole according to the present invention as shown in FIGS. 1 and 5 comprises an external sole 1 resting on the ground and at least an inner sole, interposed between said external sole and the user foot, which inner sole is composed of at least two layers a first layer directly contacting the foot, so called cleaning insole 4 and a second layer placed between said cleaning insole and said external sole 1 called intermediate layer 2, said intermediate layer 2 being at least partially composed of at least a soft layer 3 made of soft material, and said cleaning insole 4 having at least partially slots 104 said soft layer 3 penetrating therethrough and contacting the foot.

In FIG. 3 there is shown an embodiment of a soft layer 3 for example made of gel, polyurethanes, rubbers or like soft material, and/or of materials injectable according to conventional injection methods, helping in absorbing micro-shocks when walking, said soft layer extends only for a part of the plan dimensions of the inner sole, the remaining, complementary part of said inner sole being made of a stiffer material layer, and it makes a mounting insole 5 shown in FIG. 5, helping in giving a greater strength to the footwear.

Such as shown in FIG. 4 on said cleaning insole 4 slots 104 are made, in practice holes, putting in contact said soft layer 3 with the foot, as an alternative or in combination, both due to pressure exerted by the foot on said layer and to a soft and deformable material like gel, adapting to slots and penetrating therein.

According to a first preferred embodiment said soft layer 3 is provided as extending only for a part of the plan dimensions of the inner sole, the remaining, complementary part of said inner sole being made of a stiffer material layer, and it makes a mounting insole 5 shown also in FIG. 6.

According to an alternative embodiment said soft layer 3 is provided extending for all the plan dimensions of the inner sole, such as shown in FIG. 11.

As it can be noted in FIG. 6 and in FIG. 11 slots 104 are provided on said cleaning insole 4 substantially coinciding with said soft layer 3 or with a part of the soft layer 3. Moreover in order to follow plantar reflexology principles it is possible to provide said slots 104 arranged according to any predetermined pattern in the area of said cleaning insole 4 substantially coinciding with said soft layer. Thus such as shown also in FIG. 7 said soft layer contacts the foot through slots 104 in order to provide an advantageous slight massage of the foot in preferred areas.

According to a preferred embodiment shown in FIGS. 3, 4, 5, 6 said soft layer 3 is provided extending substantially in the tip area of the foot from the bending area of the foot to substantially the end of said inner sole corresponding to the foot tip, generating a greater comfort in the foot area that when walking moves away from the ground as the last one and upon which the effort pushing forwards the body is applied when walking.

According to further advantageous alternatives there are provided two, three or more soft layers, for example put one beside the other, particularly preferably there are provided two soft layers 3, 3′ a first soft layer 3 from the bending area of the foot to substantially the end of said inner sole corresponding to the foot tip, and a second soft layer 3′ at the area of the plantar arch, and slots 104 are made on the cleaning insole from the bending area of the foot to substantially the end of said inner sole corresponding to the foot tip, and/or at the plantar arch area, according to any pattern. Thus even side area of the plantar arch benefits from the slight massage due to the presence of the soft layer 3′.

According to an alternative embodiment shown in FIG. 11 the intermediate layer 2 is composed of at least a single layer of soft material, so called soft layer 3, extending substantially without interruption along the plan dimensions of said inner sole and said intermediate layer 2 at least partially along all the plan dimensions of said inner sole has a further layer stiffer than the soft layer 3 placed under said soft layer 3, so called mounting insole 5.

In this case the stiff mounting insole and the soft layer are not complementary one with respect to the other in covering the sole bottom-, but they are overlayed in order to form areas of the sole bottom with different functions: some softer areas providing the presence of the soft layer and so with anti-stress and slight massaging effect, and other stiffer areas, providing the presence only of the mounting insole and possibly of the cleaning insole, intended to strengthening the sole according to the present invention. Therefore the mounting insole 5 extends along all the plan dimensions of said inner sole and/or along part of the plan dimensions of said inner sole.

Particularly according to a preferred solution the mounting insole 5 extends along the part of the plan dimensions of said inner sole substantially coinciding with the area of the inner sole from the bending area of the foot to the foot heel. Thus the micro-shock when the foot rests is absorbed and it generates an advantageous slight massage for the user foot.

Such as shown in FIG. 11 slots 104 are also made on the cleaning insole from the bending area of the foot substantially to the end of said inner sole corresponding to the foot tip, and/or at the plantar arch area, according to any pattern, particularly such to provide advantages provided by particular massages in predetermined foot areas, as provided by known plantar reflexology.

However it is possible to provide slots 104 made on all said cleaning insole, possibly by providing a different arrangement of slots with respect to the plan dimensions of the sole, such to have areas with a greater density of slots and areas with a lower density of slots, actually generating a slight selective massage and that can be possibly adapted depending on different requirements of different users and/or different type of use of the footwear. It is possible to provide said slots 104 on all or part of the plan dimensions of said inner sole and to be arranged according to any predetermined pattern still due to reasons related to plantar reflexology and/or to different requirements of different users and/or to different type of use of the footwear.

According to a preferred embodiment shown in FIGS. 9 and 10 there is provided a footwear sole with anti-stress and slight massaging effect comprising an external sole 1 resting on the ground and at least an inner sole, interposed between said external sole and the user foot wherein said inner sole has one or more padding or cushioning areas 9. Particularly such as shown it is preferred for one of said cushioning areas to be made at the bending area of the foot, that is substantially in the foot metatarsus area and that directly contacts the foot, while the other one cushioning area 9′ is made at the foot heel, and particularly it is placed under said inner sole, at the part opposite to the inner sole one contacting the foot. Padding or cushioning areas are advantageously preferably made of anti-shock material like Poron® (licensed trademark) and/or of gel, polyurethanes, rubbers or like soft materials, such to optimally absorb micro-shocks when walking. Particularly said cushioning area 9 provided at the bending area of the foot is provided to be in direct contact with the foot, such to make at best its absorbing shock functions and comfort functions, at the same time enlarging the resting area of the foot during bending when walking, and making a resting area that is not only larger but also softer than prior art footwears.

According to a preferred embodiment variant shown in FIG. 8 cushioning areas 9, 9′ are provided in combination with a soft layer 3 as described above.

According to further variant embodiments slots 104 are made as different one with respect to the other with regard to shape and size depending on the fact of corresponding with different areas of the foot sole, or slots 104 are made identical one with respect to the other as regards shape and size.

Particularly as shown also in FIG. 2 slots 104 have a circular plan shape, since it has been found that the material of the soft layer 3 penetrates in a more uniform way inside slots and the slight massage has a better effect, such as shown in FIG. 7.

It has been further found that a further advantageous result can be obtained when said padding or cushioning area/areas are made of anti-shock material like Poron® (licensed trademark), and/or are made of gel, polyurethanes, rubbers or like soft materials.

In order to make the footwear more soft and comfortable it is further provided for the footwear, as shown in FIG. 7, to have a seam between said inner sole and the footwear lining, preferably in the area corresponding to the area from the bending area of the foot substantially to the end of said inner sole corresponding to the foot tip. Thus various parts of the footwear that are not engaged one with respect to the other are free to slide one with respect to the other making the footwear still softer and increasing comfort.

A further characteristic of the present invention is that the described and shown footwear sole with anti-stress and slight massaging effect is completely in contact with all the foot sole when walking. By the soft layer and/or the cushioning areas the sole according to the present invention is always in contact with the foot sole of the user, during all walking steps, for example avoiding the plantar arch to be moved away from the sole, and thus avoiding the subsequent micro-shock corresponding to the contact of the plantar arch with the sole that occurs when walking. If the plantar arch moves away from the sole when the foot raises, when the foot is again in contact with the ground the plantar arch again contacts the sole, actually causing a micro-shock affecting the comfort felt by the user. Advantageously this effect is avoided by arranging a footwear sole such as described and shown above.

According to a further advantageous characteristic of the present invention it is possible to provide said inner sole to be movable from said external sole and to be used individually for adding an anti-stress and/or slight massaging inner sole in already manufactured footwear, in order to increase comfort, or said inner sole to be made together with the footwear in order to produce a footwear with anti-stress and/or slight massaging effect.

According to further preferred embodiments the soft layer 3 projects by a predetermined amount from said slots coming into contact with the foot, such as shown in FIG. 7, or the soft layer 3 does not project from said slots, and it contacts the foot projecting from said slots only when walking, due to pressure exerted by the foot.

In addition the present invention can be applied also to a method for manufacturing a footwear comprising following steps, with reference to FIG. 7,

A—providing a footwear shell 90 made of at least an external vamp 98 and an inner lining 99

B—providing an external sole

C—providing an inner sole, said inner sole being made of at least a first layer directly in contact with the foot, so called cleaning insole 4,

D—providing a second layer of the inner sole, so called intermediate layer at least partially composed of a layer of soft material, called soft layer 3,

E—providing slots 104 on said cleaning insole 4 at least partially along the plan dimensions of the insole corresponding to the soft layer 3 through which said soft layer comes in contact with the foot,

F—assembling said first and said second layer to form said inner sole by sewing and/or gluing and/or joining and/or chemical physical bonding.

G—assembling said inner sole with said external sole and with said footwear shell 90 by sewing and/or gluing and/or joining and/or chemical physical bonding.

In order to make this process further advantageous following steps are provided:

A—providing a footwear shell 90 composed of at least an external vamp 98 and a inner lining 909

B—providing an external sole

C—providing an inner sole, said inner sole being made of at least a first layer directly contacting the foot, so called cleaning insole 4,

D—providing slots 104 on said cleaning insole 4 at least partially along the plan dimensions of the insole corresponding to the soft layer 3

E—assembling said inner sole with said external sole at peripheral edges by sewing and/or gluing and/or joining and/or chemical physical bonding.

F—between said inner sole and said external sole, in areas of the inner sole wherein said slots are provided injecting a soft material, such as gel, polyurthanes, rubbers or like soft materials, making a second layer of the inner sole

G—assembling said inner sole and/or said external sole and/or said footwear sole 90 by sewing and/or gluing and/or joining and/or chemical physical bonding.

Advantageously according to this process the soft or tender material is injected by means of known injection processes inside the partially manufactured sole, generating a considerable saving in costs and manufacturing time.

In order to make the footwear more strong the step of providing a footwear shell 90 made of at least en external vamp 98 and an inner lining 99 and a reinforcement section 97 interposed between said external vamp 98 and said inner lining 99 can be provided.

Advantageously in order to make softer the footwear and to increase comfort it is possible to provide sewing and/or gluing and/or joining and/or chemical physical adhering said lining with said soft layer at the peripheral area of the inner sole from the bending area of the foot to substantially the end of said inner sole corresponding to the foot tip and/or to provide to leave the external vamp 98 and the inner vamp 99 mechanically disengaged for at least a part or for all remaining parts of their surface extension. Thus various parts of the footwear that are not mechanically engaged are free to slide, giving an advantage to comfort felt by the user.

Moreover since it is provided to make also a sole that can be inserted in an already manufactured footwear, also a method and a process comprising following steps are object of the present invention:

a—providing an external sole

b—providing an inner sole, said inner sole being made of at least a first layer directly contacting the foot, so called cleaning insole,

c—providing a second layer of the inner sole, so called intermediate layer at least partially composed of at least a layer of soft material, called soft layer,

d—providing slots on said cleaning insole at least partially along the plan area of the insole corresponding to the soft layer through which said soft layer comes in contact with the foot,

e—assembling said first and said second layer to form said inner sole by sewing and/or gluing and/or joining and/or chemical physical bonding.

f—assembling said inner sole with said external sole by sewing and/or gluing and/or joining and/or chemical physical bonding.

Particularly according to a preferred variant embodiment there is provided a process for manufacturing a sole characterized in that it comprises following steps:

a—providing an external sole

b—providing an inner sole, said inner sole being made of at least a first layer directly contacting the foot, so called cleaning insole,

c—providing slots on said cleaning insole at least partially along the plan dimensions of the insole corresponding to the soft layer,

f—assembling said first and said second layer at peripheral edges by sewing and/or gluing and/or joining and/or chemical physical bonding.

g—between said inner sole and said external sole, in areas of the inner sole wherein said slots are provided injecting a soft material, such as gel, polyurthanes, rubbers or like soft materials, making a second layer of the inner sole

h—assembling said inner sole and/or said external sole by sewing and/or gluing and/or joining and/or chemical physical bonding.

Even in this case, like the process for manufacturing the footwear, the step injecting the soft or tender material allows a considerable saving as regards manufacturing time and costs.

Referring to FIGS. 12 to 13 it is possible to note a top plan view of an footbed 40 according to the present invention, that in a preferred but not limitative embodiment can be overlapped to an insole 50 inside the footwear.

The footbed 40 is shown in a preferred embodiment in FIG. 17 and FIG. 18, particularly in FIG. 17 there is shown a plan view of the upper layer 41 of the footbed according to the present invention, while in FIG. 18 there is shown a plan view of the lower layer 42 of the footbed according to the present invention, wherein the lower layer 42 is made of two different parts 42′ and 42″, while the upper layer is as one piece.

The upper layer 41 in this preferred embodiment is made as one piece, and it can comprise areas with a greater thickness, for example areas 141 at the foot sole and heel, in order to increase comfort of the footbed.

The upper layer 41 is intended to be positioned inside the footwear faced towards the user foot, such as shown in section views in FIGS. 19, 20, and 21, while the lower layer 42, made of two parts 42′ and 42″ is intended to be faced towards the ground, and/or to rest or to be glued or fastened to a cleaning insole such as shown in FIG. 13 inside the footwear.

Such as shown the footwear footbed with anti-stress effect comprising a first lower layer 42 and a second upper layer 41 and wherein said first 42 and said second layer 41 are overlapped to form said footbed 40, said first and said second layer being made of soft materials.

Particularly the ratio between density of the first lower layer 42 and said second upper layer 41 is such that:

(lower layer density/upper layer density)=0.8-0.9

particularly it is preferred that

(lower layer density/upper layer density)=0.85(+/−0.01)

Such density ratio produces a considerable comfort to the user foot, the combination of these two layers 41 and 42 having such density ratio cause an optimal absorption of shocks due to walking action and at the same time it produces also a pleasant massage to the foot sole of the user.

Particularly the upper layer has a density greater than the lower layer and a sort of buffering capacity, and that allows the footwear to adapt to the shape of the user foot and also to adapt to particular stresses exerted by the user foot on the footbed when walking.

That is achieved by means of an upper layer 41, that is advantageously made all or partially of a synthetic material, and preferably of a synthetic material based on polyurethane, characterized in that it has specific chemical-physical characteristics making it particularly comfortable and strong.

Preferable characteristics of said upper layer are described in tab. 1.

TABLE 1

| Test | | TG | | | |
|---|---|---|---|---|---|
| properties | Unity | method | Soft | Medium | Hard |
| Tensile | kPa | DIN | 140-200 | 200-300 | 300-400 |
| strength | | 53571 | | | |
| Elongation | % | DIN | 900-700 | 700-400 | 400-250 |
| at break | | 53571 | | | |
| Shore A | / | DIN | 0-5 | 5-10 | 10-20 |
| | | 53505 | | | |
| Shore L | / | DIN | 5-14 | 14-25 | 25-40 |
| | | 53519 | | | |

| | | | |
|---|---|---|---|
| Density | Kg/m³ | DIN | 1006 |
| | | 53479 | |

Particularly these characteristics makes possible for the synthetic material based on polyurethane of the upper layer 41 to have following characteristics: dimensionally stable, good absorption of shear forces, good buffering capacity, good pressure distribution, non-irritating to the skin, high elasticity, high shock absorbing power, it adheres well with glue, unplasticised and light.

Buffering capacity of this particular type of upper layer 41 described above is such that the upper layer adapts itself to the shape of the user foot, shaping itself about the foot as it were, but it has such a density to allow a considerable comfort of the user foot, while maintaining elasticity suitable to comfort. So, at each step, or even only due to weight resting on the footbed, the footbed shapes itself around the foot and it allows the footbed to adhere to all the foot sole of the user. The buffering capacity of the upper layer is realized in an elastic recovery of the upper layer, that is not immediate as the elastic recovery of the lower layer, but is has a dormant time allowing to feel a considerable comfort above all in more stressed areas.

The lower layer 42 that is the layer intended to be faced towards the footwear sole and/or towards the ground when walking is preferably made of synthetic material, particularly synthetic rubber, so called TR rubber adding pharmaceutical oils.

TR rubber is known to person skilled in the art and substantially it is a thermoplastic, synthetic rubber with a lower cost and good mechanical characteristics, in this present invention it is preferred to use TR rubber with a SHORE hardness from 5 to 8 degrees.

Particularly the lower layer advantageously has the following characteristics of tab. 2:

TABLE 2

| Technical | | Measurement | Average |
|---|---|---|---|
| characteristics | Method | unit | value |
| Density | DIN 53479 | gr/cm³ | 0.86 +/− 0.01 |
| | Method A | | |
| Tensile | DIN 53504 | Kg/cm² | 14 +/− 2 |
| Strenght (at | Test samp. S1 3 | | |
| break) | | | |
| Elongation (at | Din 53504 | % | 1050 +/− 50 |
| break) | test samp S1 3 | | |
| | Mm thickness | | |
| | trac sp. 500 | | |
| Melt Flow Index | DIN 53735 | Gr/10 min. | 200 value |
| | | | approx |
| Rebound | DIN 53512 | % | 15 +/− 4 |
| resilience | | | |
| Hardness | Din 53505 | Shore A | 5 +/− 2 |
| Compression set | ISO 815:1991 | 70° C. 24 h % | 40 +/− 5 |
| | (E) | | |
| | | 55° C. 24 h % | 5 +/− 2 |
| | | 55° C. 48 h % | 5 +/− 2 |
| | | 55° C. 72 h % | 5 +/− 2 |

Each person, different as regards physical conformation and way of walking, weight and dimensions, exerts on the footwear a different distribution of forces when walking and the presence of an footbed 40 according to the present invention allows the footwear to adapt itself to each different distribution of weights and pressures due to different users.

According to an advantageous characteristic of the present invention the lower layer 42 is discontinuous, and it is at the foot sole area 42″ and heel area 42′, and it has one or more slots 142, while the upper layer 41 is continuous or at least partially continuous in areas corresponding to said lower layer 42.

Particularly slots 142 in the lower layer are advantageous for comfort, since they produce a kind of escape for the upper layer 41 when it is pressed during the walking action or movement by the user, and it squashes on the lower layer 42.

Advantageously the presence of slots 142 in the lower layer 42 allow the upper layer 41 to project through said slots 142 towards the part opposite to the user foot, for example towards the insole 50 increasing pliability of footbed areas 40 and so of the footwear wherein said footbed 40 is mounted, wherein pressure and forces due to walking action are exerted.

When the user rests the weight on the footbed 40, the upper layer 41 gives way and it compresses itself downwardly, meeting the lower layer 42, which lower layer 42 in turn is elastically compressed downwardly, and it produces an escape, at least partially, by slots 142 for elastic deformation of the upper layer 41.

Thus the user feels a greater comfort and softness.

Referring to FIGS. 19, 20 and 21 it is possible to provide a gap between said upper layer and said lower layer, that is an air cushioning area 79, helping in increasing comfort.

Said air cushion 79 makes also a kind of further comfortable elastic effect for the user foot, the air cushion 79 produces a sort of enveloping effect when the footwear having the footbed according to the present invention is worn. When the footwear is not worn elastic properties of said first and said second layer cause the air cushion 79 to have the greatest volume position. As soon as the user wears the footwear provided with said footbed 40 with the air cushion 79, he feels the foot contacting the footbed, which at first adapts to the foot shape giving a part or all the air volume of the air cushion by pressing the air cushion area 79 due to weight.

Such considerable comfort and elasticity feeling is carried on and accentuated by the presence of the two different materials of the first and second layer of the footbed 40, which materials having above characteristics, particularly the different density and the above preferred ratio between densities, allow the footbed to have a double effect, it damps shocks and micro-shocks due to walking, and while keeping a kind of buffering capacity for foot areas of the individual user, it allows the user to feel a comfort that is considerably greater than prior art footbeds and footwears, which have a considerable elasticity degree, but do not have buffering capacity and a ratio between softness and buffering capacity as achieved by the present invention due to different materials composing the footbed, having among other things different densities, and cooperating to reach the aim overcoming drawbacks of known footwears.

It is also possible to provide such as shown in FIGS. 14, 15, 21, 24 to provide a further cushion for absorbing shocks 47 in the area substantially corresponding to the bending area of the foot, in order to optimize the comfort of the user.

Particularly the preferred area in an embodiment shown in FIG. 16 is the area 241 provided at the bending area of the foot on the footbed 40 and preferably obtained on the upper layer 41, and in a preferred embodiment the area is provided in the lower face of the upper layer, as to say between said upper and said lower layer.

In another preferred embodiment not shown, the cushion 47 for absorbing shocks, or shock absorber 47, is provided facing the ground or the insole itself, being substantially mounted in a reverse position with respect to the cushion 47 for absorbing shocks shown in FIG. 21.

The cushion 47 for absorbing shocks may be made of one of the two materials described above composing the upper layer 41 and/or the lower layer 42 or of conventional materials and it may be made integral with said layer 41 or said layer 42 or it may be applied subsequently. In the shown embodiment the cushion for absorbing shocks 47 is applied in position 241 at the side of the layer 41 faced towards the user foot, so to remain in contact with the user foot during each walking step.

Preferably the cushion for absorbing shocks 47 is made of the same material than the lower layer and it is applied below said upper layer, in the area substantially coinciding with the bending area of the foot immediately before to the plantar arch one 241.

The footbed according to the present invention is preferably intended to be mounted in a footwear and to be overlapped to a mounting insole, tipically made of texon and/or fibres, which mounting insole is overlapped to the footwear sole, and it may be covered by a conventional cleaning insole, which cleaning insole directly contacts the user foot.

The footbed 40 according to the present invention may be inserted during the process for making a footwear or it may be applied also in already manufactured footwears, when the footwear is completed.

As an alternative the footbed 40 according to the present invention can be mounted also on already manufactured footwears, or as an alternative to or in combination with the mounting insole and/or cleaning insole if they are not present.

Such as shown in FIGS. 19 and 20, the upper layer preferably has a greater thickness at the longitudinal axis of the development of the footbed 40, in order to adhere and to be squeezed above all firstly at the most projecting area of the user foot, the foot having an opposite shape. Thus the central area of the foot is better protected, where there is a considerable amount of soft tissues, that are subjected at a greater extent to micro-shocks effects due to walking, causing an early weariness feeling to the user foot.

The footbed 40 according to the present invention can be a part of the so called inner sole of the footwear, being integrated in said inner sole or being a part thereof.

In combination the footbed according to the present invention may have one or more characteristics described above according to the present invention, and such as shown in FIGS. 1 to 11.

It is also possible, according to a further embodiment variant to position the lower layer on the upper layer, allowing the layer 41 to come out through slots 142 towards the user foot instead of coming out towards the mounting insole when the two layers are squeezed, such as in the shown embodiment.

Thus a massage of the sole bottom is achieved which is definitely more incisive and can be felt by the user at a greater extent, since the layer 41 coming out from slots directly contacts the foot or the cleaning insole that in case is overlapped to the footbed according to the present invention.

There is provided also a solution where the footbed 40 according to the present invention and a cleaning insole 4 having at least one, preferably more than one slots 104 such as shown in FIG. 2 are combined together.

The footbed 40 according to the present invention may completely composed or be a part of the intermediate layer 2 and/or of the soft layer 3 shown in FIGS. 1 to 11.

Moreover the present invention applies also to a process for making a footwear comprising a footbed 40 according to the above comprising following steps:

A—providing a footwear shell made of at least an external vamp and an inner lining

B—providing an external sole intended to contact the ground

C—providing a footbed 40 made of at least an upper layer and at least a lower layer, according to what described above

D—assembling said first and said second layer of said footbed to form said footbed by sewing and/or gluing and/or joining and/or chemical physical bonding.

G—assembling said footbed 40 with said external sole and with said shell 90 of the footwear by sewing and/or gluing and/or joining and/or chemical physical bonding.

It also possible to provide to have a cleaning and/or mounting insole 50, in this case it is sufficient to add steps to above listed method steps:

H—assembling said footbed 40 with said mounting insole 50 and/or said external sole and/or said footwear shell 90 by sewing and/or gluing and/or joining and/or chemical physical bonding.

I—assembling said footbed 40 with said cleaning insole by sewing and/or gluing and/or joining and/or chemical physical bonding

1. Footwear sole with anti-stress and slight massaging effect comprising an external sole (1) resting on the ground and at least an inner sole, interposed between said external sole and the user foot, which inner sole is composed of at least two layers a first layer directly contacting the foot, so called cleaning insole (4) and a second layer placed between said cleaning insole and said external sole (1) called intermediate layer (2),
characterized in that
said intermediate layer (2) is at least partially composed of at least a soft layer (3) made of soft material,
said cleaning insole (4) having at least partially slots (104) through which said soft layer (3) penetrates and contacts the foot.

2. Sole according to the preceding claim characterized in that said soft layer (3) extends only for a part of the plan dimensions of the inner sole, the remaining, complementary part of said inner sole being made of a stiffer material layer, and it makes a mounting insole (5).

3. Sole according to claim 2, characterized in that said slots (104) on said cleaning insole (4) are made at the area of the cleaning insole (4) substantially coinciding with said soft layer (3).

4. Sole according to the preceding claim, characterized in that said slots (104) are placed according to any predetermined pattern in the area of said cleaning insole (4) substantially coinciding with said soft layer.

5. Sole according to one or more of claims 2 to 4 characterized in that said soft layer (3) substantially extends in the area of the foot tip from the bending area of the foot to substantially the end of said inner sole corresponding to the foot tip.

6. Sole according to one or more claims 2 to 5, characterized in that two, three or more soft layers are provided.

7. Sole according to the preceding claim, characterized in that preferably there are provided two soft layers (3, 3′) a first soft layer (3) from the bending area of the foot to substantially the end of said inner sole corresponding to the foot tip, and a second soft layer (3′) at the area of the plantar arch.

8. Sole according to the preceding claim characterized in that said slots (104) are made on the cleaning insole from the bending area of the foot to substantially the end of said inner sole corresponding to the foot tip, and/or at the plantar arch area, according to any pattern.

9. Sole according to claim 1 characterized in that said intermediate layer (2) is composed of at least a single layer of soft material, so called soft layer (3), extending substantially without interruption along the plan dimensions of said inner sole.

10. Sole according to claim 9 characterized in that said intermediate layer (2) at least partially along all the plan dimensions of said inner sole has a further layer stiffer than the soft layer (3) placed under said soft layer (3), so called mounting insole (5).

11. Sole according to claim 10, characterized in that said mounting insole (5) extends all along the plan dimensions of said inner sole.

12. Sole according to claim 10 characterized in that said mounting insole (5) extends along a part of the plan dimensions of said inner sole.

13. Sole according to claim 12 characterized in that said mounting insole (5) extends along the part of the plan dimensions of said inner sole substantially coinciding with the area of the inner sole from the bending area of the foot to the foot heel.

14. Sole according to the preceding claim characterized in that said slots (104) are made on the cleaning insole from the bending area of the foot to substantially the end of said inner sole corresponding to the foot tip, and/or at the plantar arch area, according to any pattern.

15. Sole according to claim 9 characterized in that said slots (104) are made on all said cleaning insole.

16. Sole according to one or more of the preceding claims characterized in that said slots (104) made on all or part of said cleaning insole have different arrangement with areas with a greater density of slots and areas with a lower density of slots.

17. Sole according to one or more of the preceding claims characterized in that said slots (104) on all or part of the plan dimensions of said inner sole, are arranged according to any predetermined pattern.

18. Footwear sole with anti-stress and slight massaging effect comprising an external sole (1) resting on the ground and at least an inner sole, interposed between said external sole and the user foot,
characterized in that
said inner sole has one or more padding or cushioning areas (9).

19. Sole according to the preceding claim characterized in that at least one of said cushioning area (9) is made at the bending area of the foot.

20. Sole according to the preceding claim characterized in that said cushioning area (9) made at the bending area of the foot directly contacts the foot.

21. Sole according to claim 18 characterized in that at least one of said cushioning area (9′) is made at the foot heel.

22. Sole according to the preceding claim characterized in that said cushioning area (9′) made at the foot heel is placed under said inner sole, at the part opposite to the inner sole one contacting the foot.

23. Sole according to one or more of claims 1 to 17 characterized in that it provides one or more of padding or cushioning areas according to one or more of claims 18 to 22.

24. Sole according to one or more of the preceding claims characterized in that said slots (104) are made different one with respect to the other with regard to shape and size depending on the fact of corresponding with different areas of the foot sole.

25. Sole according to one or more of the preceding claims characterized in that said slots (104) are made identical one with respect to the other as regards shape and size.

26. Sole according to one or more of the preceding claims characterized in that said slots (104) have a circular plan shape.

27. Sole according to one or more of the preceding claims characterized in that said soft layer is made of gel, polyurethanes, rubbers or like soft materials.

28. Sole according to one or more of the preceding claims characterized in that said padding or cushioning area/areas are made of anti-shock material like Poron® (licensed trademark), and/or are made of gel, polyurethanes, rubbers or like soft materials.

29. Sole according to one or more of the preceding claims, characterized in that said soft layer is made of materials injectable according to conventional injection methods.

30. Sole according to one or more of the preceding claims, characterized in that it has a seam between said inner sole and the footwear lining, preferably in the area corresponding to the area from the bending area of the foot substantially to the end of said inner sole corresponding to the foot tip.

31. Sole according to one or more of the preceding claims characterized in that said soft layer (3) projects by a predetermined amount from said slots coming into contact with the foot

32. Sole according to one or more of the preceding claims characterized in that said soft layer (3) does not project from said slots, and it contacts the foot projecting from said slots only when walking, due to foot pressure.

33. Footwear sole with anti-stress and slight massaging effect comprising an external sole (1) resting on the ground and at least an inner sole, interposed between said external sole and the user foot
characterized in that
said inner sole is completely in contact with all the foot sole during walking.

34. Sole according to the preceding claim characterized in that it has one or more of characteristics of claims 2 to 30.

35. Process for manufacturing a footwear characterized in that it comprises following steps:
A—providing a footwear shell (90) made of at least an external vamp (98) and an inner lining (99)
B—providing an external sole
C—providing an inner sole, said inner sole being made of at least a first layer directly in contact with the foot, so called cleaning insole (4),
D—providing a second layer of the inner sole, so called intermediate layer at least partially composed of a layer of soft material, called soft layer (3),
E—providing slots (104) on said cleaning insole (4) at least partially along the plan dimensions of the insole corresponding to the soft layer (3) through which said soft layer comes in contact with the foot,
F—assembling said first and said second layer to form said inner sole by sewing and/or gluing and/or joining and/or chemical physical bonding.
G—assembling said inner sole with said external sole and with said footwear shell (90) by sewing and/or gluing and/or joining and/or chemical physical bonding.

36. Process for manufacturing a footwear characterized in that is comprises following steps:
A—providing a footwear shell (90) composed at least of an external vamp (98) and a inner lining (99)
B—providing an external sole
C—providing an inner sole, said inner sole being made of at least a first layer directly contacting the foot, so called cleaning insole (4),
D—providing slots (104) on said cleaning insole (4) at least partially along the plan dimensions of the insole corresponding to the soft layer (3)
E—assembling said inner sole with said external sole at peripheral edges by sewing and/or gluing and/or joining and/or chemical physical bonding.
F—between said inner sole and said external sole, in areas of the inner sole wherein said slots are provided injecting a soft material, such as gel, polyurthanes, rubbers or like soft materials, making a second layer of the inner sole
G—assembling said inner sole and/or said external sole and/or said footwear shell (90) by sewing and/or gluing and/or joining and/or chemical physical bonding.

37. Process for manufacturing a footwear according to one or more claims 35, 36 characterized in that it provides the step of providing a footwear shell (90) made of at least en external vamp (98) and an inner lining (99) and a reinforcement section (97) interposed between said external vamp (98) and said inner lining (99).

38. Process for manufacturing a footwear according to one or more of claims 35 to 37 characterized in that it provides sewing and/or gluing and/or joining and/or chemical physical adhering said lining with said soft layer at the peripheral area of the inner sole from the bending area of the foot to substantially the end of said inner sole corresponding to the foot tip.

39. Process for manufacturing a footwear according to one or more of claims 35 to 38 characterized in that it provides to leave the external vamp (98) and the inner vamp (99) mechanically disengaged for at least a part or for all remaining parts of their surface extension.

40. Process for manufacturing a footwear according to one or more of claims 35 to 39 characterized in that it provides one or more characteristics of claims 1 to 34.

41. Process for manufacturing a sole characterized in that it comprises following steps:
a—providing an external sole
b—providing an inner sole, said inner sole being made of at least a first layer directly contacting the foot, so called cleaning insole,
c—providing a second layer of the inner sole, so called intermediate layer at least partially composed of at least a layer of soft material, called soft layer,
d—providing slots on said cleaning insole at least partially along the plan area of the insole corresponding to the soft layer through which said soft layer comes in contact with the foot,
e—assembling said first and said second layer to form said inner sole by sewing and/or gluing and/or joining and/or chemical physical bonding.
f—assembling said inner sole with said external sole by sewing and/or gluing and/or joining and/or chemical physical bonding

42. Process for manufacturing a sole characterized in that it comprises following steps:
a—providing an external sole
b—providing an inner sole, said inner sole being made of at least a first layer directly contacting the foot, so called cleaning insole,
c—providing slots on said cleaning insole at least partially along the plan area of the insole corresponding to the soft layer,
f—assembling said first and said second layer at peripheral edges by sewing and/or gluing and/or joining and/or chemical physical bonding.
g—between said inner sole and said external sole, in areas of the inner sole wherein said slots are provided injecting a soft material, such as gel, polyurthanes, rubbers or like soft materials, making a second layer of the inner sole
h—assembling said inner sole and/or said external sole by sewing and/or gluing and/or joining and/or chemical physical bonding.

43. Footwear footbed with anti-stress effect
characterized in that
it comprises a first lower layer (42) and a second upper layer (41) and wherein said first (42) and said second (41) layer are at least partially overlapped to form said footbed (40), said first (42) and said second (41) layer being made of soft materials.

44. Footbed according to the preceding claim, characterized in that the ratio between density of the first lower layer (42) and said second upper layer (41) is
(lower layer density/upper layer density)=0.8-0.9
said second layer (41) being intended to be positioned inside the footwear faced towards the user foot, and said first lower layer (42) being intended to be faced towards the ground.

45. Footbed according to one or more of claims 43, 44 characterized in that the ratio between density of said first lower layer (42) and said second upper layer (41) is
(lower layer density/upper layer density)=0.85(+/−0.01).

46. Footbed according to one or more of the preceding claims 43 to 45 characterized in that said upper layer (41) has a density greater than the lower layer and/or buffering capacity,

47. Footbed according to one or more of claims 43 to 46 characterized in that said upper layer (41) is made at least partially of synthetic material, and preferably of synthetic material based on polyurethane.

48. Footbed according to one or more of claims 43 to 47 characterized in that said upper layer (41) has one or more of the following characteristics: dimensionally stable, good absorption of shear forces, good buffering capacity, good pressure distribution, non-irritating to the skin, high elasticity, high shock absorbing power, it adheres well with glue, unplasticised and light.

49. Footbed according to one or more of claims 43 to 48 characterized in that said upper layer (41) has one or more of the characteristics of tab. 1
TABLE 1

| Test | | TG | | | |
|---|---|---|---|---|---|
| properties | Unity | method | Soft | Medium | Hard |
| Tensile | kPa | DIN | 140-200 | 200-300 | 300-400 |
| strength | | 53571 | | | |
| Elongation | % | DIN | 900-700 | 700-400 | 400-250 |
| at break | | 53571 | | | |
| Shore A | / | DIN | 0-5 | 5-10 | 10-20 |
| | | 53505 | | | |
| Shore L | / | DIN | 5-14 | 14-25 | 25-40 |
| | | 53519 | | | |
| | | | |
|---|---|---|---|
| Density | Kg/m³ | DIN | 1006 |
| | | 53479 | |

50. Footbed according to one or more of claims 43 to 49 characterized in that said lower layer (42), that is the layer intended to be faced towards the footwear sole and/or towards the ground when walking, is preferably made of synthetic material, particularly synthetic rubber, so called TR rubber.

51. Footbed according to one or more of claims 43 to 50 characterized in that said lower layer (42) is made of TR rubber adding pharmaceutical oils.

52. Footbed according to one or more of claims 43 to 51 characterized in that said lower layer (42), is made of thermoplastic TR rubber with a SHORE hardness from 2 to 9 degrees, particularly from 3 to 8 degrees, preferably 5+/−2 degrees.

53. Footbed according to one or more of claims 43 to 52 characterized in that said lower layer (42) has one or more of characteristics of tab. 2:
TABLE 2

| Technical | | Measurement | Average |
|---|---|---|---|
| characteristics | Method | unit | value |
| Density | DIN 53479 | gr/cm³ | 0.86 +/− 0.01 |
| | Method A | | |
| Tensile | DIN 53504 | Kg/cm² | 14 +/− 2 |
| Strenght (at | Test samp. S1 3 | | |
| break) | | | |
| Elongation (at | Din 53504 | % | 1050 +/− 50 |
| break) | test samp S1 3 | | |
| | Mm thickness | | |
| | trac sp. 500 | | |
| Melt Flow Index | DIN 53735 | Gr/10 min. | 200 value |
| | | | approx |
| Rebound | DIN 53512 | % | 15 +/− 4 |
| resilience | | | |
| Hardness | Din 53505 | Shore A | 5 +/− 2 |
| Compression set | ISO 815: 1991 | 70° C. 24 h % | 40 +/− 5 |
| | (E) | | |
| | | 55° C. 24 h % | 5 +/− 2 |
| | | 55° C. 48 h % | 5 +/− 2 |
| | | 55° C. 72 h % | 5 +/− 2 |

54. Footbed according to one or more of claims 43 to 53 characterized in that said lower layer (42) is discontinuous, and it is at the foot sole area (42″) and heel area (42′), while the upper layer (41) is continuous or at least partially continuous in areas corresponding to said lower layer (42).

55. Footbed according to one or more of claims 43 to 54 characterized in that said lower layer (42) has one or more slots (142).

56. Footbed according to one or more of claims 43 to 55, characterized in that said slots (142) produce a kind of escape for the upper layer (41) when said upper layer (41) is pressed during the walking action or movement by the user, and it squashes against the lower layer (42).

57. Footbed according to one or more claims 43 to 56 characterized in that said lower layer (42) is made of two distinct parts (42′ and 42″), said upper layer being as a single piece.

58. Footbed according to one or more of claims 43 to 57n characterized in that said upper layer (41) has a variable thickness along its extension and particularly it comprises areas with greater thickness.

59. Footbed according to one or more of claims 43 to 58 characterized in that said upper layer (41) comprises areas (141) with a greater thickness at the foot sole and heel.

60. Footbed according to one or more of claims 43 to 59 characterized in that between said upper layer and said lower layer there is a gap, that is an air cushion area (79).

61. Footbed according to one or more of claims 43 to 60 characterized in that said air cushion (79) produces a kind of elastic effect and/or envelopping effect when the footwear is worn.

62. Footbed according to one or more of claims 43 to 61 characterized in that when the footwear is not worn said air cushion (79) is in its greatest volume position, and when the footwear is worn said air cushion (79) is pressed giving a part or all air volume of the air cushion (79) and thus resulting at minimum volume.

63. Footbed according to one or more of claims 43 to 62 characterized in that it further provides a cushion for absorbing shocks (47) in the area substantially corresponding to the bending area of the foot, in order to optimize the user comfort.

64. Footbed according to one or more of claims 43 to 63 characterized in that said cushion (47) for absorbing shocks is made of one of the materials composing said upper layer (41) and/or said lower layer (42) and/or of conventional materials.

65. Footbed according to one or more of claims 43 to 64 characterized in that said cushion (47) is made integral with said upper layer (41) and/or said lower layer (42).

66. Footbed according to one or more of claims 43 to 65 characterized in that said cushion (47) is applied on or below said upper layer, in the area substantially coinciding with the bending area of the foot immediately before the plantar arch one (241).

67. Footbed according to one or more of claims 43 to 66 characterized in that said footbed is preferably mounted in a footwear and overlapped on a mounting insole, made of texon and/or fibre, which mounting insole is overlapped to the footwear external sole, and said footbed is covered by a cleaning insole, which cleaning insole directly contacts the user foot.

68. Footbed according to one or more of claims 43 to 67 characterized in that aid footbed (40) is overlapped on a mounting insole (50) inside the footwear.

69. Footbed according to one or more claims 43 to 68 characterized in that said upper layer (41) preferably has a greater thickness at the longitudinal axis of the development of the footbed (40).

70. Footbed according to one or more claims 43 to 68, characterized in that said footbed (40) is a part of the so called inner sole of the footwear, according to one or more claims 1 to 42 being integrated to said inner sole or being a part thereof.

71. Footbed according to one or more of claims 43 to 70 characterized in that it has one or more of characteristics of the sole according to one or more claims 1 to 42.

72. Footbed according to one or more claims 43 to 71 characterized in that the lower layer is placed on the upper layer, allowing the layer (41) to come out through slots (142) towards the user foot when the two layers are squeezed.

73. Footbed according to one or more claims 43 to 72 characterized in that the footbed (40) is combined together with a cleaning insole (4) havine at least one, preferably more than one slot (104) according to one or more claims 1 to 42.

74. Footbed according to one or more claims 43 to 73 characterized in that it is at least part of said intermediate layer (2) and/or soft layer (3) according to one or more claims 1 to 42.

75. Process for making a footwear comprising a footbed 40 according to one or more claims 43 to 74 characterized in that is comprises following steps:
A—providing a footwear shell made of at least an external vamp and an inner lining
B—providing an external sole intended to contact the ground
C—providing a footbed (40) made of at least an upper layer and at least a lower layer, according to what described above
D—assembling said first and said second layer of said footbed to form said footbed by sewing and/or gluing and/or joining and/or chemical physical bonding.
G—assembling said footbed (40) with said external sole and with said shell (90) of the footwear by sewing and/or gluing and/or joining and/or chemical physical bonding.

76. Process for making a footwear according to the preceding claim and further providing a cleaning insole and/or mounting insole (50),
comprising following steps:
H—assembling said footbed (40) with said mounting insole (50) and/or said external sole and/or said footwear shell (90) by sewing and/or gluing and/or joining and/or chemical physical bonding.
I—assembling said footbed (40) with said cleaning insole by sewing and/or gluing and/or joining and/or chemical physical bonding.
